SEQUENCE LISTING (2 sequences)

SEQ ID NO: 1 - protein, 12 aa, Artificial Sequence
  Synthetic Peptide
HSFKWLDSPRLR

SEQ ID NO: 2 - protein, 12 aa, Artificial Sequence
  Synthetic Peptide
SGVYKVAYDWQH

(19) United States
(12) Patent Application Publication
(10) Pub. No.: US 2019/0269771 A1
(43) Pub. Date: Sep. 5, 2019

(54) PEPTIDE MIMOTOPE THAT INDUCES AN IMMUNE RESPONSE AGAINST MYCOBACTERIUM TUBERCULOSIS LIPOARABINOMANNAN (LAM)

(71) Applicant: THE BOARD OF REGENTS OF THE UNIVERSITY OF TEXAS SYSTEM, Austin, TX (US)

(72) Inventor: Michael U. Shiloh, Dallas, TX (US)

(21) Appl. No.: 16/131,655

(22) Filed: Sep. 14, 2018

Related U.S. Application Data
(60) Provisional application No. 62/559,278, filed on Sep. 15, 2017.

(51) Int. Cl. A61K 39/04; A61K 9/00; A61K 39/39; A61P 31/06

(52) U.S. Cl. CPC A61K 39/04; A61K 9/0019; A61K 9/0043; A61P 31/06; A61K 39/39

(57) ABSTRACT

The present invention concerns methods and compositions for treating or preventing infection or dissemination of the bacterium Mycobacterium tuberculosis and for stimulating an immune response against the bacteria. In certain embodiments, the methods and compositions involve anti-LAM peptides or mimotopes. In some embodiments, the methods and compositions involve vaccine compositions. In additional embodiments, the present invention concerns peptide sequences and their use in the development of therapeutics, detection assays or vaccines against M. tuberculosis.

### CROSS REFERENCE TO RELATED APPLICATION

The present application claims priority to U.S. Application Ser. No. 62/559,278 filed Sep. 15, 2017; the entire disclosure of which is hereby incorporated by reference.

### BACKGROUND

### I. Field of the Invention

The present invention relates generally to the fields of vaccinology, immunology, pathology, bacteriology and molecular biology. More particularly, it concerns methods and compositions involving Mycobacterium tuberculosis peptide mimotopes which can be used to invoke an immune response against the bacteria. The present invention also relates to compositions, methods of preparation and use of such compositions for diagnostic and detection purposes.

### II. Background

Mycobacterium tuberculosis, the causative agent of tuberculosis, is a major cause of morbidity and mortality worldwide. It is an ancient disease that, although known to man since the Middle Ages, remains a major problem in the present day.

Notwithstanding a recent World Health Organization (WHO) tuberculosis report indicating that major progress has been made towards the global reduction of tuberculosis (Principi N, Esposito S. Tuberculosis (Edinb). 2015; 95:6-13), the global clinical, social, and economic burden of tuberculosis remains high. About one-third of the population worldwide is infected with Mycobacterium tuberculosis while nearly 9 million cases of active tuberculosis are reported annually. In 2015 there were an estimated 10.4 million new tuberculosis cases and 1.8 million tuberculosis deaths overall, including 1.0 million cases and 170,000 deaths among children (Global Tuberculosis Report. World Health Organization. 2016; available on the world wide web at who.int/tb/publications/global_report/en/)

An effective vaccine for M. tuberculosis remains lacking. The bacterium is a highly successive pathogen and has an extraordinarily complex cell wall with multiple potential immunogenic targets beyond LAM such as mycolic acids, peptidoglycan, arabinomannan (AM), lipomannan (LM), and phosphatidylinositol (PI) mannosides (PIMs), and the lipids phthiocerol dimycocerosate (PDIM), sulfolipid-1 (SL-1), and phenolic glycolipid (PGL) (Jankute et al., Annu Rev Microbiol. 2015; 69:405-23; and Bailo et al., Biochem Pharmacol. 2015; 96:159-67). Indeed, vaccination of either guinea pigs (Hamasur et al. Vaccine. 2003; 21:4081-93) or mice (Prados-Rosales et al. PLoS Pathog. 2017; 13:e1006250) with mycobacterial AM conjugated to immunogenic peptides affords protection from tuberculosis-induced mortality and dissemination during experimental infection. Antibodies against either AM (Yu et al., Clinical and vaccine immunology: CVI, 2012; 19:198-208) or LAM (Baumann et al., J Infect. 2014; 69:581-9) are detectable in the serum of M. tuberculosis infected humans, indicating that the molecules themselves are immunogenic during infection.

To date, the Bacillus Calmette Guerin (BCG) vaccine remains the only licensed vaccine for the prevention of tuberculosis. The vaccine is primarily administered in geographic areas where tuberculosis is endemic, as the protection afforded by BCG is limited to prevention of disseminated disease in children (Roy et al., BMJ. 2014; 349:g4643; and Mangtani et al. Clinical infectious diseases: an official publication of the Infectious Diseases Society of America. 2014; 58:470-80). BCG neither prevents initial infection in children, nor provides any significant protection in adults (Roy et al., 2014; and Mangtani et al., 2014). To address this deficiency, several new vaccines are now in various stages of development (Kaufmann et al., Int J Infect Dis. 2016). Although multiple innovative approaches have been proposed and tried, progress has been slow, and only one major candidate has completed a Phase IIb efficacy trial, with disappointing results (Tameris et al. Lancet. 2013; 381:1021-8). Thus, there remains an urgent need to develop novel vaccination strategies for M. tuberculosis.

A highly successful method for vaccination against both bacterial and viral pathogens has been targeting critical cell surface molecules such as the capsular polysaccharide of pneumococcus or the hemagglutinin of influenza for host antibody production. Because M. tuberculosis is enveloped by a thick carbohydrate and lipid-rich cell wall, it has not been amenable to similar vaccination strategies. Further, the prevailing paradigm in tuberculosis biology has been that antibodies play a minimal role in controlling infection since M. tuberculosis has a predominantly intracellular lifestyle. However, accumulating data suggests that anti-mycobacterial antibodies can be protective in models of M. tuberculosis infection (Achkar et al., Immunological reviews. 2015; 264:167-81; and Jacobs et al., Tuberculosis (Edinb). 2016; 101:102-13.). In fact, passive immunization with antibodies against either the dormancy protein alpha-crystallin or the cell wall component lipoarabinomannan (LAM) (Teitelbaum et al., Proc Natl Acad Sci USA. 1998; 95:15688-93.) is protective following an aerosol challenge in mice. Importantly, the currently used intradermal BCG vaccine does not induce secretory IgA production, which may in part explain its inability to provide robust protection against M. tuberculosis infection in adults. Since M. tuberculosis infection occurs via an aerosol route, an ideal vaccine would generate a robust immunologic response in the airway. Moreover, such a vaccine should be inexpensive and easily delivered in resource-limited settings.

Recently, there has been significant interest in using adenovirus as a vaccine vector for M. tuberculosis antigens (Wang et al. J Immunol. 2004; 173:6357-65.). However, the host immune response to adenovirus has limited this approach (Hartman et al., Virus research. 2008; 132:1-14; and Ahi et al., Curr Gene Ther. 2011; 11:307-20). To that end, the baculovirus Autographa californica multicapsid nucleopolyherovirus (AcMNPV) may serve as an improved vaccine vector 71. as it can be delivered via an aerosol route to effectively transduce mammalian cells (Hsu et al., Biotechnol Bioeng. 2004; 88:42-51; and Hu YC. Adv Virus Res. 2006; 68:287-320). Importantly, a very useful characteristic of AcMNPV is that it naturally infects insects but does not cause disease in mammals, as it replicates poorly within mammalian cells (Hu, 2006). After aerosol infection, AcMNPV enters the cytoplasm of macrophages and dendritic cells, thus eliciting both humoral and cell mediated immunity characterized by a vigorous Th1 response (Abe et al., J Immunol. 2003; 171:1133-9; Wilson et al., Vaccine. 2008; 26:2451-6; and Jordan et al., Curr Protoc Immunol. 2009; Chapter 2:Unit 2 15). To date, AcMNPV has been used to successfully vaccinate against influenza (Abe et al., 2003) and to generate a robust response against a malaria protein (Strauss et al. Mol Ther. 2007; 15:193-202). In the case of influenza, when recombinant AcMNPV expressing full-length influenza hemagglutinin was used to inoculate mice intranasally, mice were protected from lethal challenge with influenza due to immunoglobulin production and stimulation of a potent innate and cell mediated immune response (Abe et al., 2003). Likewise, a recombinant baculovirus both displayed the malaria circumsporozoite protein on its surface and drove intracellular expression of the protein within antigen presenting cells induced antibody, CD4 and CD8 responses in vitro (Strauss et al., 2007). Most animals lack pre-existing antibodies against baculovirus, and baculovirus is stable at room temperature, which could eventually allow it to be utilized as a human vaccine without need for refrigeration.

The mycobacterial cell wall is a protein, lipid and carbohydrate rich structure thought to be involved in the virulence of M. tuberculosis. The major constituents are the mycolic acid lipids, arabinomannan (AM) and lipoarabinomannan (LAM) polysaccharides, and 89 cell wall associated proteins (Brennan P J. Tuberculosis (Edinb). 2003; 83:91-7). Previously, when various components of the cell wall have been delivered subcutaneously, vaccination has been equivalent to the live attenuated Mycobacterium bovis bacillus Calmette-Guerin (BCG) vaccine. However, direct vaccination with lipids or cell wall sugars may be deleterious, as they have potent immunomodulatory activities. An alternative to using immunomodulatory lipids or sugars is to utilize mimotopes, short peptides that can elicit protective antibodies against non-peptide antigens (Pirofski LA. Trends Microbiol. 2001; 9:445-51). Such mimotopes behave as surrogate antigens, inducing an immune response that is cross-reactive with the native antigen. Peptide mimotopes have been used successfully to vaccinate against the Cryptococcus neoformans capsular polysaccharide (Fleuridor et al., J Immunol. 2001; 166:1087-96), and importantly, mimotopes targeting M. tuberculosis LAM have been reported (Gevorkian et al. The Biochemical journal. 2005; 387:411-7; Sharma et al., Clinical and vaccine immunology: CVI. 2006; 13:1143-54; and Barenholz et al., J Med Microbiol. 2007; 56:579-86). However, the ability of mimotopes to protect against M. tuberculosis infection has not been tested to date.

Development of an improved tuberculosis vaccine has been a primary goal for decades (Kaufmann et al., 2016). Though many candidate gene approaches have been tried, to date none have succeeded in providing either enhanced early protection against infection or in preventing dissemination better than the currently available BCG vaccine (Kaufmann et al., 2016). Thus, new approaches and vaccination strategies are needed. Several previous studies have reported the development of peptide mimotopes for the mycobacterial LAM antigen (Table 1), though none of those reports tested the ability of LAM mimotopes to afford protective immunity against M. tuberculosis infection

### SUMMARY OF THE DISCLOSURE

LAM is a major cell wall component of M. tuberculosis that is thought to serve as a modulin with immunoregulatory and anti-inflammatory effects. LAM is reported to allow the bacterium to maintain its survival by undermining the host resistance and acquired immune responses. This has posed a deterrent for the development of effective prophylactic and curative measures against the disease. Disclosed herein are new mimotope peptides against M. tuberculosis LAM, such as SEQ ID NO:1, and a M. tuberculosis vaccination strategy combining anti-LAM peptide mimotope with a delivery system.

Embodiments of the present invention include methods for eliciting an immune response against Mycobacterium tuberculosis in a recipient or a subject comprising administering to the subject an effective amount of a composition comprising a peptide of SEQ ID NO:1 or variants thereof. In certain aspects, the composition is formulated as a pharmaceutically acceptable composition. A pharmaceutically acceptable composition can include, for example, a vaccine, a therapeutic, a drug, a small molecule, or an antibody against M. tuberculosis. In certain aspects of the method, the composition is given more than once to the subject. In some aspects of the method, the composition is administered orally, parentally, subcutaneously, intradermally, intramuscularly, intramucosally, intravenously, or intranasally.

Embodiments of the present invention include methods for eliciting an immune response against Mycobacterium tuberculosis in a recipient or a subject comprising administering to the subject an effective amount of a composition comprising a peptide of SEQ ID NO:1 or variants thereof and an additional composition comprising another M. tuberculosis antigen. In a further embodiment, the additional composition comprises a Bacillus Calmette Guerin (BCG) vaccine. In other aspects of the method of eliciting an immune response, the compositions are administered in the same formulation. In some aspects, the compositions are administered within 24, 12, 6, or 3 hours from each other or any amount of time in between. In some aspects, the compositions are administered within one hour, within 30 minutes from each other or within any time frame in between. In some aspects the compositions are administered at the same time.

Embodiments of the present invention include methods for stimulating in a subject a protective or a therapeutic immune response against Mycobacterium tuberculosis in the subject comprising administering to the subject an effective amount of a composition comprising a 1) peptide of SEQ ID NO:1 or variants thereof 2) a nucleic acid molecule encoding SEQ ID NO:1 or variants thereof or 3) any permutation of SEQ ID NO:1 described herein.

Embodiments of the current invention includes methods of preparing an immunoglobulin for use in prevention or treatment of M. tuberculosis infection comprising the steps of immunizing a recipient with a composition comprising SEQ ID NO:1 or variants thereof and isolating the immunoglobulin from the recipient. In some aspects of the methods, the recipient is a mammal including but not limited to mouse, rabbit, human, etc.

One of skill in the art will recognize that individual substitutions, additions, or deletions to a peptide, which alters, adds, or deletes a single or more amino acid(s) is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid. Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Some embodiments are directed to methods for treatment or prevention of M. tuberculosis infection comprising a step of administering to a patient an effective amount of pharmaceutical preparation of an immunoglobulin that binds M. tuberculosis LAM, wherein the immunoglobulin is raised against SEQ ID NO:1 or sequences comprising SEQ ID NO:1, such as by methods described herein or by any method that is known to a person of skill in the art.

Other embodiments are directed to a use of the compositions and pharmaceutical preparations described herein of the immunoglobulins that bind SEQ ID NO:1 or variants thereof in the manufacture of a medicament for the treatment or prevention of M. tuberculosis infection.

Some embodiments include vaccines comprising a pharmaceutically acceptable composition having an isolated SEQ ID NO:1 or variants thereof described herein, or any other combination or permutation of peptides or sequences described herein, wherein the composition is capable of stimulating an immune response against M. tuberculosis. In certain aspects a protein or peptide of the invention is linked (covalently or non-covalently) to an adjuvant. In some aspects, the adjuvant is chemically conjugated to the protein.

In some embodiments, a vaccine composition is a pharmaceutically acceptable composition having a recombinant nucleic acid encoding all or part of SEQ ID NO:1, or encoding any other combination or permutation of peptide(s) or mimotopes described herein, wherein the composition is capable of stimulating an immune response against M. tuberculosis.

Embodiments of the current invention includes methods for detecting the presence of M. tuberculosis (or antibodies against M. tuberculosis LAM) in a subject, the method comprising: a. obtaining a biological sample from the subject, b. detecting anti-LAM antibodies in the sample by using any composition containing an immunoglobulin (or polypeptides, proteins and/or peptides) described herein; and c. determining the presence or absence of M. tuberculosis.

In addition to the use of immunoglobulins, polypeptides, proteins, and/or peptides, as well as antibodies binding these immunoglobulins, polypeptides, proteins, and/or peptides, to treat or prevent infection as described above, the present invention contemplates the use of these immunoglobulins, polypeptides, proteins, peptides, and/or antibodies in a variety of ways, (including the detection of the presence of M. tuberculosis) to diagnose an infection, in a subject including but not limited to a patient.

Some embodiments are directed to an immunoglobulin prepared by the methods described herein. In some aspects, the immunoglobulin is a polyclonal antibody or a monoclonal antibody.

In addition, the present invention contemplates the use of these immunoglobulins polypeptides, proteins, peptides, and/or antibodies in a variety of ways, including the detection of the presence of M. tuberculosis to diagnose an infection, whether in a subject including but not limited to a patient. Also provided are embodiments for the use of immunoglobulins, polypeptides, and/or peptides, as well as antibodies binding these polypeptides, proteins, and/or peptides, to treat or prevent infection.

Embodiments of the present invention also include methods of diagnosing the presence of M. tuberculosis (or antibodies against M. tuberculosis, or LAM) in a subject, the method comprising: a. obtaining a biological sample from the subject and b. detecting anti-LAM antibodies in the sample by using a composition comprising SEQ ID NO:1, so as to determine the presence or absence of M. tuberculosis. In some embodiments of the methods, SEQ ID NO:1 is conjugated or complexed to other molecules. Such molecules include but are not limited to capture antibodies, or enzymes or reporter molecules, etc.

In certain embodiments, the current invention provides isolated peptides and peptide mimotopes, and variants thereof having at least about 80% sequence identity or similarity to SEQ ID NO:1. In some embodiments, the peptide can have at least about 60%, 65%, 70%, 75%, 80%, 85%, 90%, 95%, 97%, 98% or 99% sequence identity to SEQ ID NO:1 or any percentage in between. In some aspects, the isolated peptides comprise at least 4, 5, 6, 7, 8, 9, 10, or 11 contiguous amino acids of SEQ ID NO:1. In certain aspects, the peptides comprise an amino acid sequence exactly that of SEQ ID NO:1 or the peptides consist of SEQ ID NO:1. In some embodiments, provided are peptides wherein an amino acid substitution is made to SEQ ID NO: 1. The substitution can be in one or more amino acids of SEQ ID NO:1 as described above. Provided are isolated peptides wherein the peptides are an anti-M. tuberculosis lipoarabinomannan (LAM) mimotopes. In some embodiments, the isolated peptides are conjugated to a moiety, a carrier protein or a vector. In some aspects the peptides are chemically conjugated to, or expressed as, a fusion protein with other proteins. For purposes of this specification and the accompanying claims, all such fused proteins are included in the definition of antibodies or an immunological portion of an antibody.

In some embodiments, the peptides of the current invention are conjugated to a recombinant baculovirus. In some aspects, the baculovirus is Autographa californica multinuclear polyhedrosis virus (AcMNPV). In some embodiments, the isolated peptides are conjugated to a keyhole limpet hemocyanin (KLH) protein.

Other embodiments are directed to a use of peptides in the manufacture of a medicament for the treatment or prevention of M. tuberculosis infection. In alternate embodiments, the peptides or variants thereof are used in the detection of M. tuberculosis. In some embodiments the detection is the presence of the bacterium in a cell line in the lab or in a subject having an infections.

Embodiments of the current invention provide isolated nucleic acids, compositions and methods involving these nucleic acids, comprising a nucleotide sequence encoding any of the peptide described herein.

In some embodiments of the current invention, provided are compositions comprising any of the peptides described above. In some aspects, the composition is an immunogenic composition or a vaccine or a combination vaccine. In some aspect, the vaccine or the combination vaccine includes an additional M. tuberculosis vaccine such as, for example, the Bacillus Calmette Guerin (BCG) vaccine.

In some embodiments, the present invention provides for pharmaceutical compositions and preparations comprising peptides comprising SEQ ID NO:1 or variants thereof. In some aspects, the pharmaceutical compositions elicit an immune response against M. tuberculosis. In some aspects, the pharmaceutical composition includes Bacillus Calmette Guerin (BCG) vaccine. Also provided are the use of the pharmaceutical composition in the manufacture of a medicament for the treatment or prevention of M. tuberculosis infection.

In some embodiments of the current invention, provided are methods of preventing or treating M. tuberculosis infection comprising a step of administering to a subject an effective amount of the pharmaceutical compositions described here. In some aspects, the subject is at risk of developing an infection with M. tuberculosis. In some embodiments the subject is human. In some aspects, the methods of treating a subject comprise administering to the subject an effective amount of a therapeutic composition or a vaccine. In some aspects, the vaccines are developed through the use of a peptides, or peptide mimotope comprising SEQ ID NO:1 and variants thereof as described above.

In some embodiments, provided are methods of preventing or treating Mycobacterium bovis infections in animals comprising a step of administering to an animal an effective amount of a pharmaceutical composition comprising SEQ ID NO:1 or variants thereof as described above. In some embodiments, the pharmaceutical composition is a vaccine. In some embodiments the animals are livestock animals including but not limited to cows, bulls, pigs, sheep, goats, yak, bison, buffalo, oxen and horses.

In some embodiments, provided are kits for detecting the presence of antibodies against M. tuberculosis or M. tuberculosis LAM, or against M. bovis in a cell line, a subject, or an animal wherein the kit comprises a composition comprising the isolated peptide or nucleic acids or immunoglobulins or peptide mimotopes described herein. In some aspects the kits are for use in immunoassays. In some aspects are enzyme-linked immunosorbent assays (ELISA). In some embodiments of the kit, the isolated peptides in the kits act as an antigen that binds anti-LAM antibodies. In certain aspects, the kits are for vaccinating against M. tuberculosis or M. bovis infections. In other embodiments, the kits comprise vaccine composition or compositions. In some embodiments, the kits include instructions for using the kit.

It is contemplated that any method, composition, peptide or kit described herein can be implemented with respect to the treatment, detection or diagnosis of M. bovis in mammals including but not limited to livestock, cattle and other farm animals.

It is contemplated that any method or composition or peptide described herein can be implemented with respect to any other method or composition described herein and that different embodiments may be combined.

Use of the one or more compositions may be employed based on methods described herein. Certain embodiments, for example, relate to a use of a composition comprising SEQ ID NO: 1. Some embodiments relate to a use of an immunoglobulin obtained by immunizing a recipient with a composition comprising SEQ ID NO:1 or variants thereof. Yet other embodiments relate to use of a pharmaceutical composition comprising the immunoglobulin described herein. One or more of such compositions may be employed in the preparation or manufacture of medicaments for treatment or prevention of M. tuberculosis or immunoglobulins for vaccines or detection methods according to the methods described herein. Some embodiments relate to use of an isolated peptide comprising SEQ ID NO:1 or variants thereof in the manufacture of a medicament for the treatment or prevention of M. tuberculosis infection. Some embodiments relate to use of a pharmaceutical composition comprising a peptide comprising SEQ ID NO:1 (or an anti-M. tuberculosis lipoarabinomannan (LAM) peptide mimotope comprising SEQ ID NO:1) or variants thereof in the manufacture of a medicament for the treatment or prevention of M. tuberculosis infection. Additional embodiments relate to methods of treating a subject against M. tuberculosis infection, comprising administering to the subject an effective amount of a therapeutic composition of a vaccine developed through the uses described herein.

Additional use of one or more compositions may be employed in the preparation or manufacture of medicaments for treatment or prevention of M. bovis in animals, immunoglobulins for vaccines, or detection methods according to the methods described herein. Some embodiments relate to use of an isolated peptide comprising SEQ ID NO:1 or variants thereof in the manufacture of a medicament for the treatment or prevention of M. bovis infection in animals. Some embodiments relate to use of a pharmaceutical composition comprising a peptide comprising SEQ ID NO:1 (or an anti-M. tuberculosis lipoarabinomannan (LAM) peptide mimotope comprising SEQ ID NO:1) or variants thereof in the manufacture of a medicament for the treatment or prevention of M. bovis infection in animals. Additional embodiments relate to methods of treating an animal against M. tuberculosis infection, comprising administering to the animal an effective amount of a therapeutic composition of a vaccine developed through the uses described herein.

Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

In some aspects, the subject has a tuberculosis infection, is suspected of having, or is at risk of developing a tuberculosis infection. Disclosed compositions include immunogenic compositions wherein the peptide(s), antigen(s) or epitope(s) are present in an amount effective to achieve the intended purpose. More specifically, an effective amount means an amount of active ingredients necessary to stimulate or elicit an immune response, or provide resistance to, amelioration of, or mitigation of infection. In more specific aspects, an effective amount prevents, alleviates, or ameliorates symptoms of disease or infection, or prolongs the survival of the subject being treated. Determination of the effective amount is well within the capability of those skilled in the art, especially in light of the detailed disclosure provided herein. For any preparation used in the methods of the invention, an effective amount or dose can be estimated initially from in vitro studies, cell culture, and/or animal model assays. For example, a dose can be formulated in animal models to achieve a desired immune response or circulating antibody concentration or titer. Such information can be used to more accurately determine useful doses in humans.

A “subject,” “individual” or “patient” is used interchangeably herein and refers to a vertebrate, for example a primate, a mammal or a human. Mammals include, but are not limited to cattle, equines, canines, bovines, ovines, murines, rats, simians, humans, farm animals, sport animals and pets. Also intended to be included as a subject are any subjects involved in clinical research trials not showing any clinical sign of disease, or subjects involved in epidemiological studies, or subjects used as controls.

The term “providing” is used according to its ordinary meaning to indicate “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

The term “substantially the same” or “not significantly different” refers to a level of expression that is not significantly different than what it is compared to. Alternatively, or in conjunction, the term substantially the same refers to a level of expression that is less than 2, 1.5, or 1.25 fold different than the expression or activity level it is compared to.

The term “similarity” refers to a peptide that has a sequence that has a certain percentage of amino acids that are either identical with the reference peptide or constitute conservative substitutions with the reference peptides.

Moieties of the invention, such peptides, or antigens, may be conjugated or linked covalently or non-covalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.” Recombinant fusion proteins are particularly contemplated. Compositions of the invention may further comprise an adjuvant or a pharmaceutically acceptable excipient. An adjuvant may be covalently or non-covalently coupled to a peptide of the invention. In certain aspects, the adjuvant is chemically conjugated to the peptide.

“Diagnosis” may refer to the process of attempting to determine or identify a possible disease or disorder, or to the opinion reached by this process. From the point of view of statistics the diagnostic procedure may involve classification tests.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some embodiments it is contemplated that a numerical value discussed herein may be used with the term “about” or “approximately.” The term “about” or “around” is also used to indicate that a value includes the standard deviation of error for the device or method being employed to determine the value.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. “Consisting essentially of” in the context of pharmaceutical compositions of the disclosure is intended to include all the recited active agents and excludes any additional non-recited active agents, but does not exclude other components of the composition that are not active ingredients. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result.

Embodiments defined by each of these transition terms are within the scope of this invention.

The term “isolated” can refer to peptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated peptide” is a nucleic acid or protein fragment that is not naturally occurring as a fragment and/or is not typically in the functional state.

The terms “protein”, “polypeptide” and “peptide” are used interchangeably herein when referring to a gene product or functional protein.

A “modified peptide” or a “variant” refers to a peptide whose chemical structure, particularly its amino acid sequence, is altered with respect to SEQ ID NO:1. It is specifically contemplated that a modified/variant peptide may be altered with respect to its sequence but retains activity or function in some aspects such as immunogenicity.

The term “amino acid” includes naturally-occurring α-amino acids and their stereoisomers, as well as unnatural (non-naturally occurring) amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid. The term “amino acid modification” or “amino acid alteration” refers to a substitution, a deletion, or an insertion of one or more amino acids.

The term “nucleic acid,” “nucleotide” or “polynucleotide” refers to deoxyribonucleic acids (DNA) or ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with gene, cDNA, and mRNA encoded by a gene.

The term “nucleotide sequence encoding a peptide” means the segment of DNA involved in producing a peptide chain, it includes regions preceding and following the coding region (leader and trailer) involved in the transcription/translation of the gene product and the regulation of the transcription/translation, as well as intervening sequences (introns) between individual coding segments (exons).

“Percentage of sequence identity” is determined by comparing two optimally aligned sequences over a comparison window, wherein the portion of the sequence (e.g., a peptide of the invention) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

The term “percent identity” or “percent sequence identity,” in the context of describing two or more polynucleotide or amino acid sequences, refer to two or more sequences or subsequences that are the same or have a specified percentage of amino acid residues or nucleotides that are the same (for example, a variant of a peptide of interest (e.g., mimotope of interest) used in the method of this invention has at least 80% sequence identity, preferably 85%, 90%, 91%, 92%, 93, 94%, 95%, 96%, 97%, 98%, 99%, or 100% identity, to a reference sequence, e.g., a corresponding epitope or mimotope of interest), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 8 amino acids in length, or more preferably over a region that is at least 8-25 or at least 8 to 12 amino acids in length.

The terms “ameliorating,” “inhibiting,” or “reducing,” or any variation of these terms, when used in the claims and/or the specification includes any measurable decrease or complete inhibition to achieve a desired result.

The term “inhibitor” refers to a therapeutic agent that indirectly or directly inhibits the activity or expression of a protein, process (e.g. metabolic process), or biochemical pathway The term “pharmaceutical formulation” is intended to mean a composition or a mixture of compositions comprising at least one active ingredient; including but not limited to, salts, solvates, and hydrates of compounds described herein.

As used herein, “treating,” “treatment” or “therapy” is an approach for obtaining beneficial or desired clinical results. This includes the prevention of the disease, the prevention of latent infections or the reduction in the frequency of recurrence of latent infections. It also includes the reduction, the amelioration or the alleviation of symptoms, including but not limited to cough, chest pain, weight loss, fever, night sweats and/or chills. Furthermore, these terms are intended to encompass curing as well including the curing of an active infection.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The use of the word “a” or “an” when used in conjunction with the term “comprising” in the claims and/or the specification may mean “one,” but it is also consistent with the meaning of “one or more,” “at least one,” and “one or more than one.”

As used herein, a “fragment” refers to a sequence having or having at least 5, 6, 7, 8, 9, 10, or more contiguous residues of the recited SEQ ID N01, but less than the full-length of the SEQ ID NOs. It is contemplated that the definition of “fragment” can be applied to amino acid and nucleic acid fragments.

As used herein, an “antigenic fragment” refers to a fragment, as defined above, that can elicit an immune response in an animal.

As used herein, “mimotopes” are peptides mimicking protein, carbohydrates or lipid epitopes and can be generated by phage display technology. Coupled to carriers or presented in a multiple antigenic peptide form mimotopes achieve immunogenicity and induce epitope-specific antibody responses upon vaccination. Mimotopes can be peptides, such as peptides with an amino acid sequence length of at least about 8 to about 25 amino acids or more.

The term “epitope” refers to a binding site including an amino acid motif (e.g., a linear amino acid sequence or a particular three dimensional structure) which can be bound by an immunoglobulin (e.g., IgE, IgG, etc.) or recognized by a T-cell receptor when presented by an APC in conjunction within the major histocompatibility complex (MHC).

The embodiments in the Example section are understood to be embodiments of the invention that are applicable to all aspects of the invention.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. It is to be noted, however, that the appended drawings illustrate certain embodiments of the invention and therefore are not to be considered limiting in their scope.

FIGS. 1A-M Analysis of previously published LAM mimotopes. (A) Testing a polyclonal rabbit and two mouse monoclonal antibodies in a LAM ELISA. (B-E) Mimotope peptides or their scramble controls were conjugated to KLH and mimotope behavior determined by ELISA using the rabbit polyclonal anti-LAM antibody. (F-J) Mimotope peptides or their scramble controls were conjugated to KLH and mimotope behavior determined by ELISA using the mouse monoclonal CS-35 anti-LAM antibody. K-M Mice (n=5 per peptide) were vaccinated with PBS, KLH alone, or mimotope peptides or scramble controls conjugated to KLH and sera collected after 4 vaccinations. Mouse sera (1:100 dilution) was tested by ELISA against LAM (K), KLH (L) or specific peptides (M) with CS-35 as a positive control and preimmune 413 sera or mouse IgG as negative controls. One of two representative experiments is shown.

FIGS. 2A-C Analysis of novel anti-LAM mimotopes by ELISA. (A) Plates were coated with either phage alone, LAM, HS peptide-expressing phage, or SG peptide-expressing phage (10 μg/ml for each coating molecule) and binding by rabbit anti-LAM polyclonal antibody determined by ELISA. (B) Plates were coated with BSA, LAM, HS peptide-expressing phage, HS peptide alone, HS peptide conjugated to KLH, HS scramble peptide alone (HSSR) or HSSR conjugated to KLH (10 μg/ml for each coating molecule) and binding by rabbit anti-LAM polyclonal antibody determined by ELISA. (C) Plates were coated with BSA, LAM, SG peptide-expressing phage, SG peptide alone, SG peptide conjugated to KLH, SG scramble peptide alone (SGSR) or SGSR conjugated to KLH (10 μg/ml for each coating molecule) and binding by rabbit anti-LAM polyclonal antibody determined by ELISA. One of two representative experiments is shown

FIGS. 3A-B Vaccination with HS-peptide conjugated to KLH induces anti-LAM antibodies. Mice (n=5) per group were vaccinated with HS peptide conjugated to KLH (A) or HSSR peptide conjugated to KLH (B) and sera was collected after 4 vaccinations. Serum was collected from individual mice and tested for binding (1:100 dilution) to BSA, LAM, HS peptide conjugated to KLH, HSSR conjugated to KLH or KLH alone (10 μg/ml for each coating molecule) by ELISA. Shown is a scatter plot from one experiment of two. (STATS).

FIGS. 4A-D Baculovirus conjugated HS peptide is protective in a low dose aerosol tuberculosis infection model in mice. (A) Test peptide (P1 biotin) was conjugated to baculovirus and detected by anti-biotin Western blot. Lane 1 is baculovirus conjugated to P1 biotin, lane 2 is peptide alone, and lane 3 is baculovirus alone. (B) Plates were coated with BSA, LAM, HS peptide435 expressing phage, HS peptide alone, HSSR peptide alone, baculovirus alone, HS peptide conjugated to baculovirus or HSSR conjugated to baculovirus (10 μg/ml for each coating molecule) and binding by rabbit anti-LAM polyclonal antibody determined by ELISA. (C) Mice (n=5) per group were vaccinated intranasally with HS peptide conjugated to baculovirus or HSSR peptide conjugated to baculovirus and sera was collected after 4 vaccinations. Serum was collected from individual mice and tested for binding (1:100 dilution) to BSA, LAM, or baculovirus (10 μg/ml for each coating molecule) by ELISA. Shown is a scatter plot from one experiment of two. (STATS). (D) Mice (n=10 per group) vaccinated as in C with baculovirus alone, HS peptide conjugated to baculovirus, HSSR peptide conjugated to baculovirus or M. bovis var BCG were infected with 200 CFU M. tuberculosis Erdman strain and monitored for survival. *p<0.05, **<p<0.01 by Gehan-Breslow-Wilcoxon test compared to baculovirus vaccination.

### DETAILED DESCRIPTION OF ILLUSTRATIVE EMBODIMENTS

Described herein is the use of anti-LAM monoclonal antibodies to pan a phage display library and uncover novel mimotopes that can serve, for example, in the generation of vaccines against M. tuberculosis. A phage display library was panned using two monoclonal antibodies against M. tuberculosis lipoarabinomannan (LAM) and identified two peptide sequences with high antibody affinity after multiple rounds of panning. Recombinant M13 phages displaying the peptides HSFKWLDSPRLR (SEQ ID NO:1) or SGVYKVAYDWQH (SEQ ID NO:2) on the g3p minor coat protein showed strong binding affinity with both mAbs. Both peptides were able to induce an anti-LAM response when conjugated to either keyhole limpet hemocyanin (KLH) or to the baculovirus Autographa californica multicapsid nucleopolyherovirus (AcMNPV) and introduced into mice by injection or via intranasal infection, respectively. Vaccination with AcMNPV conjugated HSFKWLDSPRLR peptide delayed mortality in a mouse model of tuberculosis.

The inventor began by first testing the known peptide sequences. Surprisingly, mimotope activity could not be detected by the inventor for the published peptide sequences, neither in vitro as antigenic substrates in ELISA nor in vivo after vaccinating experimental animals.

It was thus found that a peptide mimotope against the mycobacterial cell wall component LAM was identified by panning a phage display library, this mimotope can induce an anti-LAM antibodies after vaccination either when conjugated to KLH or baculovirus, and a baculovirus-mimotope anti-LAM vaccination strategy can afford partial protection against mortality from M. tuberculosis infection.

### A. LIPOARABINOMANNAN (LAM)

A critical contributor to the ability of M. tuberculosis to evade the host immune system is its hydrophobic and complex cell wall, which is composed of peptidoglycan, arabinogalactan, mycolic acids, and glycolipids layered on top of the plasma membrane. Lipoarabinomannan (LAM), is a mannose-containing glycolipids that is an important component of the cell envelope. LAM is a polysaccharide chain containing α-D-arabinofuranosyl (Araf) and mannopyranosyl residues. It spans the mycobacterial outer membrane and the covalently linked macromolecules of the cell envelope referred to as mycolyl-arabinogalactan-peptidoglycan complex (mAGP), terminating in a phosphatidylinositol-diacylglycerol moiety that is embedded within the cytoplasmic membrane. In addition to serving as a major cell wall component, it is thought that LAM serves as a modulin by controlling the host immune and inflammatory responses. This ensures the survival of the bacterium by undermining host resistance and acquired immune responses. Immunoregulatory mechanisms include the inhibition of T-cell proliferation and of macrophage microbicidal activity via diminished IFN-γ response. Additional functions of lipoarabinomannan are thought to include the neutralization of cytotoxic oxygen free radicals produced by macrophages, inhibition of protein kinase C, and induction of early response genes.

### B. MIMOTOPES AND PHAGE DISPLAY TECHNOLOGY

Mimotopes are peptides mimicking protein, carbohydrates, or lipid epitopes that are usually generated by phage display technology. When selected by antibodies, they represent exclusively B-cell epitopes and are devoid of antigen/allergen-specific T-cell epitopes. Coupled to carriers or presented in a multiple antigenic peptide forms, mimotopes achieve immunogenicity and induce epitope-specific antibody responses upon vaccination.

Phage display technology is an advanced tool to define peptide mimotopes that mimic natural epitopes including both conformational and linear epitopes. It is one of the most powerful and widely used laboratory technique for the study of protein-protein, protein-peptide, and protein-DNA interactions. The technology is mainly based on displaying the interest protein (peptides, antibodies, scaffolds or others) on the surface of employing phage so as to be used to interrogate the constructed libraries containing millions or even billions of displayed phages. The strength of the phage technology lies in this display of up to 10⁹ different peptides in a library form enabling the selection of mimotopes in a repetitive screening procedure. Theoretically, phage display is an exogenous gene expression method which the gene encoding the interest protein is inserted into bacteriophage coat protein gene then displaying the interest protein on the phage surfaces, resulting in a connection between genotype and phenotype. A large number of protein-antibody, virus-antibody and ligand-receptor interfaces have already been mapped by the use of phage display technology.

For the creation of a peptide phage display library random oligonucleotides are inserted into the genome of the filamentous bacteriophage M13 using either the minor coat protein pIII (display of 3-5 copies/phage) or the major coat protein pVIII (display of up to 2700 copies/phage) as display system on the surface of the phages. The peptides can be presented in either linear or circular form. To construct circular peptides the sequence has to be flanked by two cysteine residues. By building a disulfide bond a constrained cycle is formed and presented on the phage surface. Beside the different structural presentations, the length of the peptides can vary from 6 to 38 amino acids, Thus, within this phage system only the presentation of relatively short peptide sequences is possible, whereas larger gene insertions of proteins or antibody fragments appear to prevent pIII and pVIII functions necessary for phage reproduction, To overcome this problem phagemid systems have been developed. The phagemid itself carries only the phage gene gill or gVIII, containing the foreign sequence, and needs a helper phage with all the necessary genes for phage production including also a copy of the wild-type gene used for display. Therefore, both recombinant and wild type proteins will be produced and incorporated into the phage particle. For pill one of five copies, and for pVIII from 1% to 30% of the 2700 copies will display the foreign protein. For the production of combinatorial antibody libraries primarily phagemid systems are used resulting in the display of ‘giant mimotopes’, most often in association with pIII. Antibody display can be performed: i) by antibody fragment (Fab) systems, including two light chain domains (variable and constant) and the variable and first constant domain of the heavy chain; ii) by single-chain variable fragment (scFv) systems where only the variable domains of each chain are presented; or iii) by single-chain Fab (scFab).

In ‘biopanning’ allergen- or antigen-specific antibodies of interest, monoclonal or polyclonal, are adsorbed on microtiter plates and incubated with a phage display library containing the complete repertoire of the respective library. Phages displaying peptide or protein domains which bind to the antibodies are caught whereas unbound phages are removed by washing steps. Bound phages are then eluted from the complexes by acidic solutions (such as HCl or glycine buffer) or by competition with the original antigen, if available. Amplified phage particles of the preceding round are used as starting material for the next panning round. Thereby, specifically interacting ligands can be amplified with great efficacy. Eluents from the biopanning rounds are-tested by ELISA or other immunological methods. An increase in the titre of phage particles specifically binding to the selection antibody during the panning rounds is a first indicator of successful selection. Subsequently, the colony screening method is used to identify specifically interacting phage clones for further analysis. Generally, a strong signal in these tests predicts good mimicry of the original antigen, but this has to be additionally proven by competition assays with the original antigen or allergen. After sequencing the most promising clones, computational matching studies can be performed using a software program rendering visualization of the location and the structural features of the epitope of interest Subsequent immunization studies with the mimotopes must prove molecular mimicry and should lead to antibodies recognizing the original allergen/antigen.

### C. VACCINES

The methods of the disclosure also include the administration of vaccines. As used herein, the term in vitro administration refers to manipulations performed on cells removed from or outside of a subject, including, but not limited to cells in culture. The term ex vivo administration refers to cells that have been manipulated in vitro, and are subsequently administered to a subject. The term in vivo administration includes all manipulations performed within a subject, including administrations.

In certain aspects of the present disclosure, the compositions may be administered either in vitro, ex vivo, or in vivo. In certain in vitro embodiments, autologous T cells are incubated with compositions of this disclosure. The cells can then be used for in vitro analysis, or alternatively for ex vivo administration.

Method aspects of the disclosure include vaccinating a subject with a variety of different immunotherapeutic compositions. In some embodiments, the methods further comprise administration of SEQ ID NO: 1 to a subject. The route of administration of the immune cell may be, for example, intratumoral, intracutaneous, subcutaneous, intravenous, intralymphatic, and intraperitoneal administrations. In some embodiments, the administration is intratumoral or intralymphatic.

The present invention includes methods for preventing or ameliorating M. tuberculosis and/or M. bovis infections. As such, the invention contemplates vaccines for use in both active and passive immunization embodiments. Immunogenic compositions, proposed to be suitable for use as a vaccine, may be prepared from immunogenic peptides comprising SEQ ID NO:1 or variants thereof as described herein. In certain aspects, antigenic material is extensively dialyzed to remove undesired small molecular weight molecules and/or lyophilized for more ready formulation into a desired vehicle.

The present invention includes peptides and compositions for preventing, eliciting an immune response against, diagnosing and detecting M. tuberculosis and/or M. tuberculosis LAM. As such, the invention contemplates vaccines for use in both active and passive immunization embodiments. Immunogenic compositions, proposed to be suitable for use as a vaccine, may be prepared from peptides of SEQ ID NO:1.

Embodiments provided include methods of preventing, vaccinating or treating Mycobacterium bovis infections in animals comprising a step of administering to an animal an effective amount of a pharmaceutical composition comprising SEQ ID NO:1 or variants thereof, such as a vaccine. In some embodiments the animals are livestock animals including but not limited to cows, bulls, pigs, sheep, goats, yak, bison, buffalo, oxen and horses.

Other options for a protein/peptide-based vaccine involve introducing nucleic acids encoding the antigen(s) as DNA vaccines. In this regard, reports described construction of recombinant vaccinia viruses expressing either 10 contiguous minimal CTL epitopes (Thomson, 1996) or a combination of B cell, cytotoxic T-lymphocyte (CTL), and T-helper (Th) epitopes from several microbes, and successful use of such constructs to immunize mice for priming protective immune responses. Thus, there is ample evidence in the literature for successful utilization of peptides, peptide-pulsed antigen presenting cells (APCs), and peptide-encoding constructs for efficient in vivo priming of protective immune responses. The use of nucleic acid sequences as vaccines is exemplified in U.S. Pat. Nos. 5,958,895 and 5,620,896.

The preparation of vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines may be conventionally administered parenterally, by injection, for example, either subcutaneously or intramuscularly. Additional formulations which are suitable for other modes of administration include suppositories and; in some cases, oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The peptides and peptide-encoding DNA constructs may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable; but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain instances, it will be desirable to have multiple administrations of the vaccine, e.g., 2, 3, 4, 5, 6 or more administrations. The vaccinations can be at 1, 2, 3, 4, 5, 6, 7, 8, to 5, 6, 7, 8, 9, 10, 11, 12 twelve week intervals, including all ranges there between. Periodic boosters at intervals of 1-5 years will be desirable to maintain protective levels of the antibodies. The course of the immunization may be followed by assays for antibodies against the antigens

1. Carriers

A given composition may vary in its immunogenicity. It is often necessary therefore to boost the host immune system, as may be achieved by coupling a peptide or polypeptide to a carrier. Exemplary and preferred carriers are keyhole limpet hemocyanin (KLH) and bovine serum albumin (BSA). Other albumins such as ovalbumin, mouse serum albumin, or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde, and bis-biazotized benzidine.

KLH (Keyhole Limpet Hemocyanin) is used extensively as a carrier protein in research and therapeutic vaccine applications. Peptides, small proteins and drug molecules of low molecular weight are not usually immunogenic. They require the aid of a carrier protein to stimulate antibody production. KLH is a very effective and popular carrier protein due to its large size, numerous epitopes, plentiful sites for antigen conjugation, and strong safety history. KLH is also well known as a safe, potent stimulator of humoral and cellular immune responses. It used in research and clinical studies as an antigen for assessing immune function and in immunotoxicology studies such as monitoring the immunosuppressive effects of drug candidates.

2. Adjuvants

The immunogenicity of peptide compositions can be enhanced by the use of nonspecific stimulators of the immune response, known as adjuvants. Suitable adjuvants include all acceptable immunostimulatory compounds, such as cytokines, toxins, or synthetic compositions. A number of adjuvants can be used to enhance an antibody response against a variant of SEQ ID NO:1 or combination contemplated herein. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Adjuvants include, but are not limited to, oil-in-water emulsions, water-in-oil emulsions, mineral salts, polynucleotides, and natural substances. Specific adjuvants that may be used include IL-1, IL-2, IL-4, IL-7, IL-12, γ-interferon, GMCSP, BCG, aluminum salts, such as aluminum hydroxide or other aluminum compound, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM), and cell wall skeleton (CWS) in a 2% squalene/Tween 80 emulsion. MHC antigens may even be used. Others adjuvants or methods are exemplified in U.S. Pat. Nos. 6,814,971, 5,084,269, 6,656,462, each of which is incorporated herein by reference.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

Examples of and often preferred adjuvants include complete Freund's adjuvant (a nonspecific stimulator of the immune response containing killed Mycobacterium tuberculosis), incomplete Freund's adjuvants, and aluminum hydroxide.

In some aspects, it is preferred that the adjuvant be selected to be a preferential inducer of either a Th1 or a Th2 type of response. High levels of Th1-type cytokines tend to favor the induction of cell mediated immune responses to a given antigen, while high levels of Th2-type cytokines tend to favor the induction of humoral immune responses to the antigen.

The distinction of Th1 and Th2-type immune response is not absolute. In reality an individual will support an immune response, which is described as being predominantly Th1 or predominantly Th2. However, it is often convenient to consider the families of cytokines in terms of that described in murine CD4+ T cell clones by Mosmann and Coffman (Mosmann, and Coffman, 1989). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as γ-interferon, IL-2, or IL-12 or genes encoding proteins involved in immune helper functions, such as B-7.

### D. FORMULATIONS AND ROUTES OF ADMINISTRATION

In some embodiments, pharmaceutical compositions are administered to a subject. Different aspects of the present invention involve administering an effective amount of a composition to a subject. In some embodiments of the present invention, anti-LAM peptide mimotopes or antigens, may be administered to a recipient to protect against infection by M. tuberculosis. Alternatively, an expression vector encoding one or more such peptides may be given to a patient as a preventative treatment. Additionally, such compounds can be administered in combination with an antibiotic or an antibacterial. Such compositions will generally be dissolved or dispersed in a pharmaceutically acceptable carrier or aqueous medium.

In addition to the compounds formulated for parenteral administration, such as those for intravenous or intramuscular injection, other pharmaceutically acceptable forms include, e.g., tablets or other solids for oral administration, time release capsules; and any other form currently used, including inhalants and the like.

The active compounds of the present invention can be formulated for parenteral administration, e.g., formulated for injection via the intravenous, intramuscular, sub-cutaneous, or even intraperitoneal routes. The preparation of an aqueous composition that contains a compound or compounds that increase the expression of an MHC class I molecule will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### E. IMMUNOGLOBULINS, ANTIBODIES AND PASSIVE IMMUNIZATION

Another aspect of the invention is a method of preparing an immunoglobulin for use in prevention or treatment of staphylococcal infection comprising the steps of immunizing a recipient or donor with the vaccine of the invention and isolating immunoglobulin from the recipient or donor. An immunoglobulin prepared by this method is a further aspect of the invention. A pharmaceutical composition comprising the immunoglobulin of the invention and a pharmaceutically acceptable carrier is a further aspect of the invention that could be used in the manufacture of a medicament for the treatment or prevention of M. tuberculosis disease. A method for treatment or prevention of M. tuberculosis infection comprising a step of administering to a patient an effective amount of the pharmaceutical preparation of the invention is a further aspect of the invention.

Inocula for polyclonal antibody production are typically prepared by dispersing the antigenic composition in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies.

The antibodies can be isolated to the extent desired by well-known techniques such as affinity chromatography (Harlow and Lane, 1988). Antibodies can include antiserum preparations from a variety of commonly used animals, e.g. goats, primates, donkeys, swine, horses, guinea pigs, rats or man.

An immunoglobulin produced in accordance with the present invention can include whole antibodies, antibody fragments or subfragments. Antibodies can be whole immunoglobulins of any class (e.g., IgG, IgM, IgA, IgD or IgE), chimeric antibodies or hybrid antibodies with dual specificity to two or more antigens of the invention. They may also be fragments (e.g., F(ab′)2, Fab′, Fab, Fv and the like) including hybrid fragments. An immunoglobulin also includes natural, synthetic, or genetically engineered proteins that act like an antibody by binding to specific antigens to form a complex.

A vaccine of the present invention can be administered to a recipient who then acts as a source of immunoglobulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat staphylococcal infection. Hyperimmune globulins of the invention are particularly useful for treatment or prevention of staphylococcal disease in infants, immune compromised individuals, or where treatment is required and there is no time for the individual to produce antibodies in response to vaccination.

An additional aspect of the invention is a pharmaceutical composition antibodies (or fragments thereof; preferably human or humanized) reactive against at least two constituents of the immunogenic composition of the invention, which could be used to treat or prevent infection M. tuberculosis. Such pharmaceutical compositions comprise monoclonal antibodies that can be whole immunoglobulins of any class, chimeric antibodies, or hybrid antibodies with specificity to two or more antigens of the invention. They may also be fragments (e.g., F(ab′)2, Fab′, Fab, Fv and the like) including hybrid fragments.

Methods of making monoclonal and polyclonal antibodies are well known in the art and can include the fusion of spleenocytes with myeloma cells (Kohler and Milstein, 1975; Harlow and Lane, 1988). Alternatively, monoclonal Fv fragments can be obtained by screening a suitable phage display library (Vaughan et al., 1998). Monoclonal antibodies may be humanized or part humanized by known methods.

### F. IMMUNOASSAYS

Embodiments of the current invention include methods of detection and immunoassays related to peptides of SEQ ID NO:1 or immunoglobulins against said peptides. Immunoassays generally include immunoblotting (e.g., Western blotting), RIA, and ELISA. More specific types of immunoassays include antigen capture/antigen competition, antibody capture/antigen competition, two-antibody sandwiches, antibody capture/antibody excess, and antibody capture/antigen excess. Methods of making antibodies are described herein and in Harlow and Lane, Antibodies: A Laboratory Manual, 1988, Cold Spring Harbor Laboratory Press, Cold Spring Harbor, N.Y., USA. Phospho-specific antibodies can be made de novo or obtained from commercial or noncommercial sources. Phosphorylation levels and/or status can also be determined by metabolically labeling cells with radioactive phosphate in the form of [.gamma.-.sup.32P]ATP or [.gamma.-.sup.33P]ATP. Phosphorylated proteins become radioactive and hence traceable and quantifiable through scintillation counting, radiography, and the like (see, e.g., Wang et al., J. Biol. Chem., 253:7605-7608 (1978)).

The present invention includes the implementation of serological assays to evaluate whether and to what extent an immune response is induced or evoked by compositions of the invention. There are many types of immunoassays that can be implemented. Immunoassays encompassed by the present invention include, but are not limited to, the Immunoassays described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo. Immunoassays generally are binding assays. Certain preferred immunoassays are the various types of enzyme linked immunosorbent assays (ELISAs) and radioimmunoassays (RIA) known in the art. For example, peptide mimotopes comprising SEQ ID NO:1 can be used as antigens in ELISA detection assays which are assays well known to a person of skill in the art.

Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### G. DIAGNOSIS OF M. TUBERCULOSIS INFECTION

In addition to the use of peptides, as well as antibodies binding these M. tuberculosis antigens or peptide mimotopes comprising SEQ ID NO: 1 or variants described herein, to prevent an infection as described above, the present invention contemplates the use of these peptides, and/or antibodies in a variety of ways, including the detection of the presence of M. tuberculosis to diagnose an infection, whether in a patient or a subject at risk of developing an infection.

Current methods to detect exposure to M. tuberculosis rely on either skin test (PPD) or blood test (IGRA), both of which require 2-3 days to complete. For diagnostic purposes, detection of anti-LAM antibodies against a readily produced peptide could be significantly more rapid. In addition, the novel methods of diagnosis provided by the current invention are more rapid and direct for detecting exposure to tuberculosis as the current tuberculosis vaccine, BCG has limited efficacy.

In accordance with the invention, a preferred method of detecting the presence of infections involves the steps of obtaining a sample suspected of being infected by one or more M. tuberculosis or strains thereof, such as a sample taken from an individual, for example, from one's blood, saliva, tissues, fluids, lungs, for example. Following isolation of the sample, diagnostic assays utilizing the peptides, and/or antibodies of the present invention may be carried out to detect the presence of M. tuberculosis, and such assay techniques for determining such presence in a sample are well known to those skilled in the art and include methods such as radioimmunoassay, western blot analysis and ELISA assays. In general, in accordance with the invention, a method of diagnosing an infection is contemplated wherein a sample suspected of being infected with M. tuberculosis has added to it the polypeptide, protein, peptide, antibody, or monoclonal antibody in accordance with the present invention, and M. tuberculosis are indicated by antibody binding to the polypeptides, proteins, and/or peptides, or polypeptides, proteins, and/or peptides binding to the antibodies in the sample.

Accordingly, antibodies in accordance with the invention may be used for the prevention of infection from M. tuberculosis (i.e., passive immunization), for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. Specific examples of the generation of an antibody to a bacterial protein can be found in U.S. Patent Application Pub. No. 20030153022, which is incorporated herein by reference in its entirety.

Any of the above described polypeptides, proteins, peptides, mimotopes and/or antibodies may be labeled directly with a detectable label for identification and quantification of staphylococcal bacteria. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

### H. DELIVERY SYSTEMS

In one insect expression system that may be used to produce the chimeric proteins of the invention, Autographa californica nuclear polyhidrosis virus (AcNPV) is used as a vector to express the foreign genes. The virus grows in Spodoptera frugiperda cells. A coding sequence may be cloned into non-essential regions (for example, the polyhedron gene) of the virus and placed under control of an AcNPV promoter (for example, the polyhedron promoter). Successful insertion of a coding sequence will result in inactivation of the polyhedron gene and production of non-occluded recombinant virus (i.e. virus lacking the proteinaceous coat coded for by the polyhedron gene). These recombinant viruses are then used to infect Spodoptera frugiperda cells in which the inserted gene is expressed. (see, e.g., Smith et al. 1983, J. Virol. 46:584; U.S. Pat. No. 4,215,051). Further examples of this expression system may be found in Ausubel et al., eds. 1989, Current Protocols in Molecular Biology, Vol. 2, Greene Publish. Assoc. & Wiley Interscience.

### I. COMBINATION THERAPIES

The compositions and related methods, particularly the administration of an anti-LAM peptide mimotope of SEQ ID NO: 1 can be also used in combination with the administration of conventional therapies and vaccinations against M. tuberculosis infections. The compositions and related methods of the present invention, particularly administration of vaccine or immunoglobulin prepared in connection with SEQ ID NO:1, including a variant peptide, can also be used in combination with the administration of traditional therapies such as antibiotics or other vaccines such as a Bacillus Calmette Guerin (BCG) vaccine. The Bacillus Calmette-Guerin (BCG) vaccine has existed for decades and is one of the most widely used of all current vaccines, reading >80% of neonates and infants in countries where it is part of the national childhood immunization programs. BCG vaccine has a documented protective effect against meningitis and disseminated TB in children. It does not prevent primary infection and, more importantly, does not prevent reactivation of latent pulmonary infection, the principal source of bacillary spread in the community. Combination with other vaccine such as the vaccines contemplated in the current disclosure could provide a more effective measure against M. tuberculosis infections. To that end, identification of anti-LAM mimotopes such as provided by the current invention, might provide even greater protection when combined with other vaccines or therapies.

In one aspect, it is contemplated that a polypeptide vaccine and/or therapy is used in conjunction with an antibacterial treatment. Alternatively, the therapy may precede or follow the other agent treatment by intervals ranging from minutes to weeks. In embodiments where the other agents and/or a proteins or polynucleotides are administered separately, one would generally ensure that a significant period of time did not expire between the time of each delivery, such that the agent and antigenic composition would still be able to exert an advantageously combined effect on the subject. In such instances, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example antibiotic therapy is “A” and the immunogenic molecule given as part of an immune therapy regime, such as an antigen, is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the immunogenic compositions of the present invention to a patient/subject will follow general protocols for the administration of such compounds, taking into account the toxicity, if any, of the current composition, or other compositions described herein. It is expected that the treatment cycles would be repeated as necessary. It also is contemplated that various standard therapies, such as hydration, may be applied in combination with the described therapy.

### J. KITS

Certain aspects concern kits containing compositions described herein or compositions to implement methods described herein.

In some embodiments, kits may be provided to evaluate the presence of M. tuberculosis or LAM or antibodies against LAM. Such kits can be prepared from readily available materials and reagents. For example, such kits can comprise any one or more of the following materials: enzymes, reaction tubes, buffers, detergent, primers and probes, ELISA reagents, antibodies, enzymes. In a particular embodiment, these kits allow a practitioner to obtain various biological samples.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

Alternatively, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits in certain aspects. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained.

A kit may also include instructions for employing the kit components as well the use of any other reagent not included in the kit. Instructions may include variations that can be implemented.

### K. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of particular embodiments, are exemplary, and are not intended as limitations on the scope of the invention. Changes therein and other uses which are encompassed within the spirit of the invention as defined by the scope of the claims will occur to those skilled in the art.

### Example 1: Testing Existing Mimotopes In Vitro and in Mice

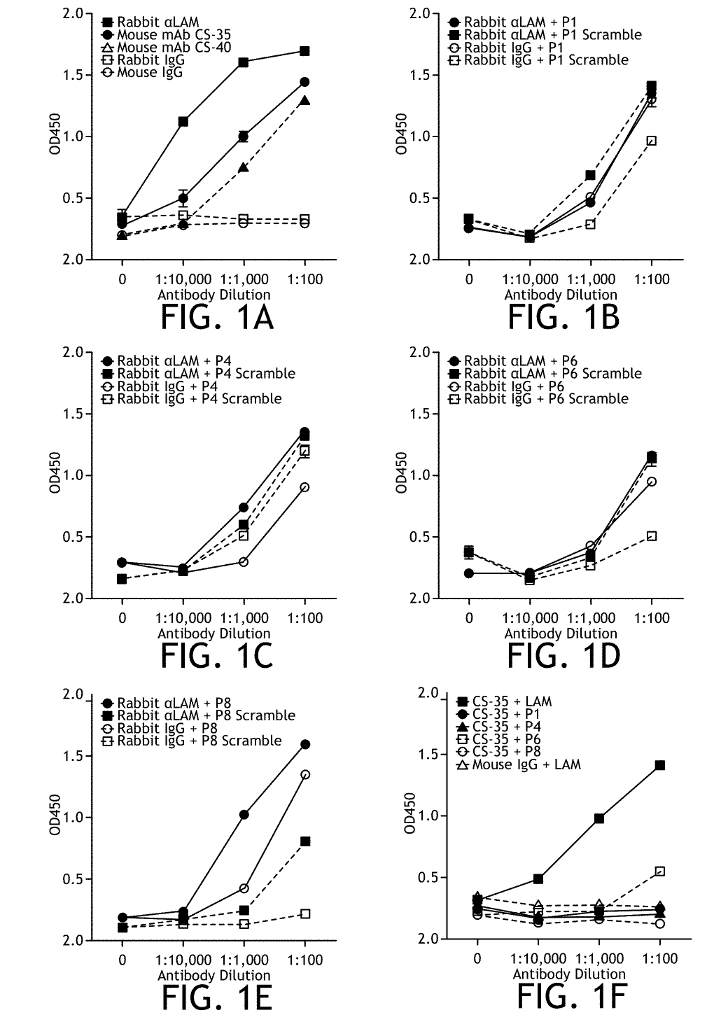

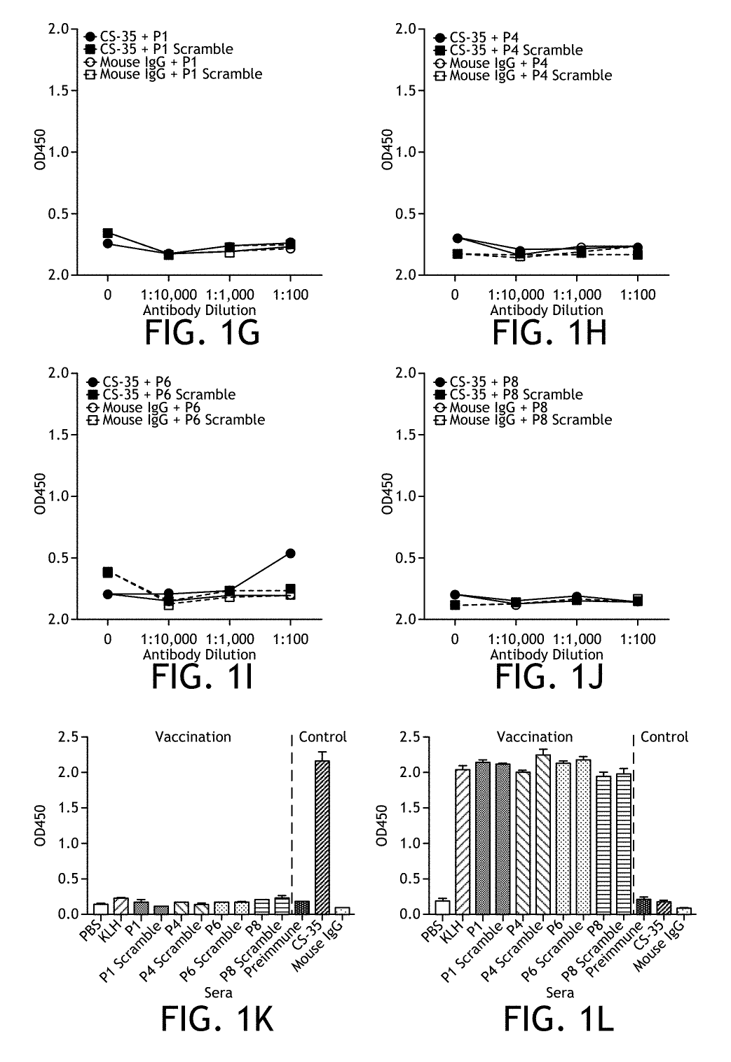

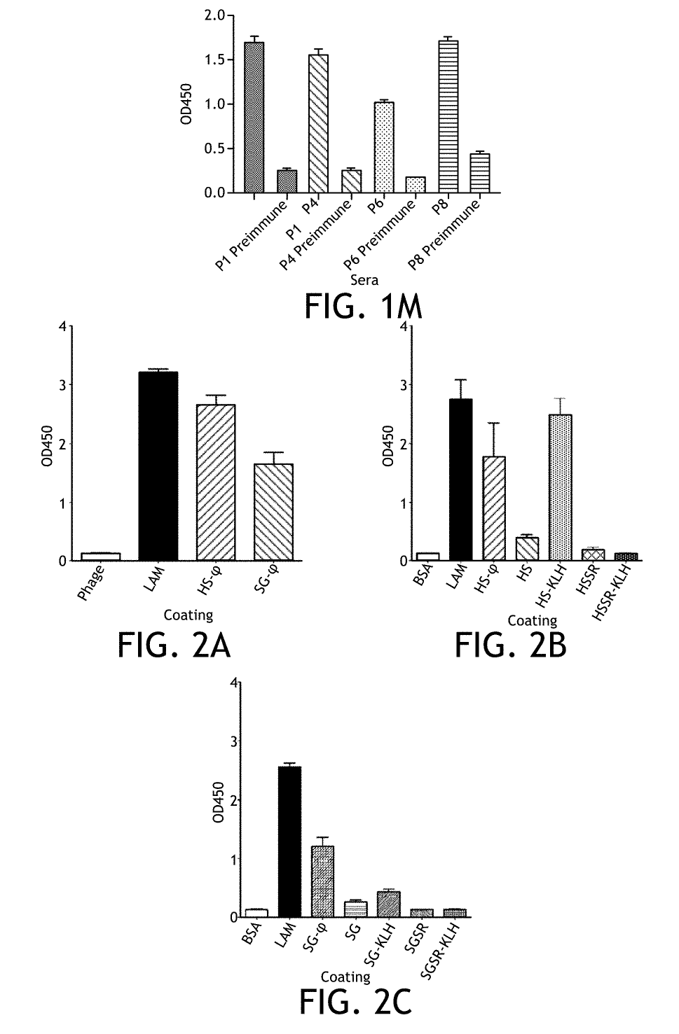

To test the ability of peptide mimotopes to induce an anti-LAM response and protect mice in vivo, either a rabbit polyclonal anti-LAM antibody or two mouse monoclonal anti-LAM antibodies (CS-35 and CS-40) were tested for their ability to bind LAM by ELISA (FIG. 1A). All three antibodies were able to bind LAM, though the rabbit polyclonal could bind at a lower titer compared to the two mouse monoclonal antibodies tested (FIG. 1A). Next 4 previously published peptides and their scrambled controls were synthesized (Table 1) (Gevorkian et al., 2005; Sharma et al., 2006; and Barenholz et al., 2007), conjugated to KLH and tested the ability of either the rabbit anti-LAM polyclonal antibody or the mouse monoclonal antibody CS-35 to bind the KLH-conjugated peptides in vitro by ELISA (FIG. 1B-J). Of the previously published peptides, only P8 could be specifically detected by the rabbit anti-LAM polyclonal antibody above control (FIG. 1E), and only at the 1:1000 dilution. Likewise, none of the peptides conjugated to KLH were bound by the CS-35 mAb (FIG. 1F-J). Next, the peptides were tested as to whether they could induce an anti-LAM immune response in mice. Outbred Swiss-Webster mice were injected 4 times with PBS, KLH alone, KLH conjugated to peptide or KLH conjugated to control peptide along with an alum adjuvant, and sera from the mice after the 4^{th} injection were collected. Using a LAM ELISA, it was found that as with the in vitro assay, none of the vaccinations with KLH-conjugated mimotope peptides induced anti-LAM antibodies at an antibody dilution of 1:100, while the CS-35 mAb showed a strong response (FIG. 1K). As expected, immunization induced a robust anti-KLH (FIG. 1L) and anti-peptide (FIG. 1M) response. Taken together, the data indicate that none of the previously identified anti-LAM mimotope peptides could be bound by anti-LAM antibodies in vitro or induce an anti-LAM mimotope response during experimental vaccination.

| Peptide | Sequence |
|---|---|
| P1 | CQEPLMGTVPIRAGGGS |
| P1 scramble | CQLGAIEMPGSPGTVGR |
| P1 biotin | CQEPLMGTVPIRAGGGSR-Biotin |
| P4 | CMSPRATI |
| P4 scramble | CIPARMTS |
| P6 | CSHRLLQTYWSSA |
| P6 scramble | CQSTSHLYASWLR |
| P8 | CISLTEWSMWYRH |
| P8 scramble | CRSWEWHSTLMYI |
| HS | CHSFKWLDSPRLR |
| HS scramble | CPHDFRLWSLSRK |
| SG | CSGVYKVAYDWQH |
| SG scramble | CGSYVVAYWDKHQ |

### Example 2: Screening Phage Display Libraries with Anti-LAM Monoclonal Antibodies CS-35 and CS-40

Since monoclonal antibodies have defined epitope specificity compared to polyclonal antibodies, biopanning with the monoclonal antibodies rather than the polyclonal antibody was pursued. Five rounds of bio-panning of a 12-mer phage display library (NEB) were conducted independently, with either CS-35 or CS-40. As expected, the number of high-affinity phages recovered increased significantly over the course of the biopanning. After the fifth panning cycle, 10 randomly selected phages per antibody were sequenced. When biopanning with CS-40 mAb, 2 peptide motifs predominated: HSFKWLDSPRLR (hereafter called 232 HS peptide after the two N-terminal amino acids OR SEQ ID NO:1) accounted for 70% ( 7/10) of the sequenced phages, while SGVYKVAYDWQH (hereafter called SG peptide or SEQ ID NO:2) accounted for 30% ( 3/10). Biopanning with CS-35 mAb revealed the predominance of only 1 peptide motif, SGVYKVAYDWQH, which was identical to the SG peptide obtained from biopanning with CS-40. To determine if the two identified peptide motifs represented false-positive target-unrelated peptides (TUP) (Bakhshinejad et al., Amino Acids. 2016; 48:2699-716), the SAROTUP (Huang et al., J Biomed Biotechnol. 2010; 2010:101932) and Mimo-DB (Huang et al. Nucleic Acids Res. 2012; 40:D271-7) databases were searched with each peptide. While neither peptide was identified as having previously been found to be a TUP using the TUPScan software, both peptides had previously been identified during other target searches. In particular, the SG peptide has been identified 5 times previously (Chen et al., Mol Pharm. 2015; 12:2180-8; Ren et al., Biochem Pharmacol. 2016; 107:91-100; and Diaz-Perlas et al., Biopolymers. 2016), while the HS peptide has only been identified once (Chen et al., 2015). Interestingly, both the SG and HS peptide were identified in the same screen for peptides that bind the calcium-independent mannose-6-phosphate receptor (Chen et al., 2015). Thus, since LAM is composed of a number of mannose residues and the calcium-independent mannose-6-phosphate receptor binds a modified mannose motif, it is feasible that both peptide mimotopes create a three-dimensional structure similar to one or more mannose residues.

### Example 3: Characterization of Mimotope Behavior of Identified Peptides

To determine if the putative mimotope peptides identified by biopanning using the mouse monoclonal antibodies can also be detected by LAM antibodies from another species, the ability of the rabbit polyclonal anti-LAM antibody to bind the putative mimotope peptides was tested. Thus, plates were coated with LAM alone as a positive control, HS-conjugated phage, SG-conjugated phage or phage alone and the magnitude of binding of the rabbit anti-LAM antibody was determined. Coating the plate with LAM yielded the greatest OD value (3.23), which was similar to coating with the HS phage (2.82) (FIG. 2A). Coating wells with an equivalent amount of SG-conjugated phage yielded the lowest response (OD 1.56).

The ability of the rabbit anti-LAM polyclonal antibody to recognize the mimotope peptides under a variety of conditions was compared. Thus, plates were coated with LAM as a positive control or HS peptide alone, HS peptide displayed on bacteriophage, HS peptide conjugated to KLH, the scrambled version of the HS peptide (HSSR) or the HSSR peptide conjugated to KLH and the magnitude of binding of rabbit polyclonal anti-LAM antibody to each hapten was determined by ELISA. The OD values were 2.8, 0.45, 1.81, and 2.56 for LAM, HS peptide alone, HS phage, and HS-KLH respectively (FIG. 2B). No binding was detected for the scramble peptide alone or when conjugated to KLH. When the experiment was repeated for the SG peptide, the OD values were 2.8, 0.32, 1.36, and 0.57 for LAM, SG peptide alone, SG phage, and SG-KLH respectively (FIG. 2C). For both peptides, coating with synthetic peptide alone did not result in significant antibody binding. In contrast, while the anti-LAM polyclonal antibody bound both HS and SG peptides displayed on bacteriophage, only the HS-peptide could be bound when conjugated to KLH. Thus, it can be concluded that both peptides require conjugation to a larger protein to be successfully recognized by anti-LAM antibody, and that the HS peptide is more consistently detected by the anti-LAM polyclonal antibody. Finally, to determine if mice can generate anti-LAM antibodies after vaccination with LAM, and if such antibodies can detect the HS-peptide, Swiss-Webster mice were vaccinated with LAM combined with alum and tested the serum against various antigens by ELISA (FIG. 2D). As expected, serum from LAM vaccinated mice robustly bound LAM. Mouse anti-LAM antibodies also bound the HS-peptide conjugated to KLH, but not the HSSR control peptide conjugated to KLH or KLH alone (FIG. 2D). Thus, it can be concluded that anti-LAM antibodies from either rabbit (polyclonal) or mouse (monoclonal or polyclonal) bind the HS-peptide when it is conjugated to a carrier molecule.

### Example 4: Mimotope Activity of KLH-Conjugated HS-Peptide in Vivo

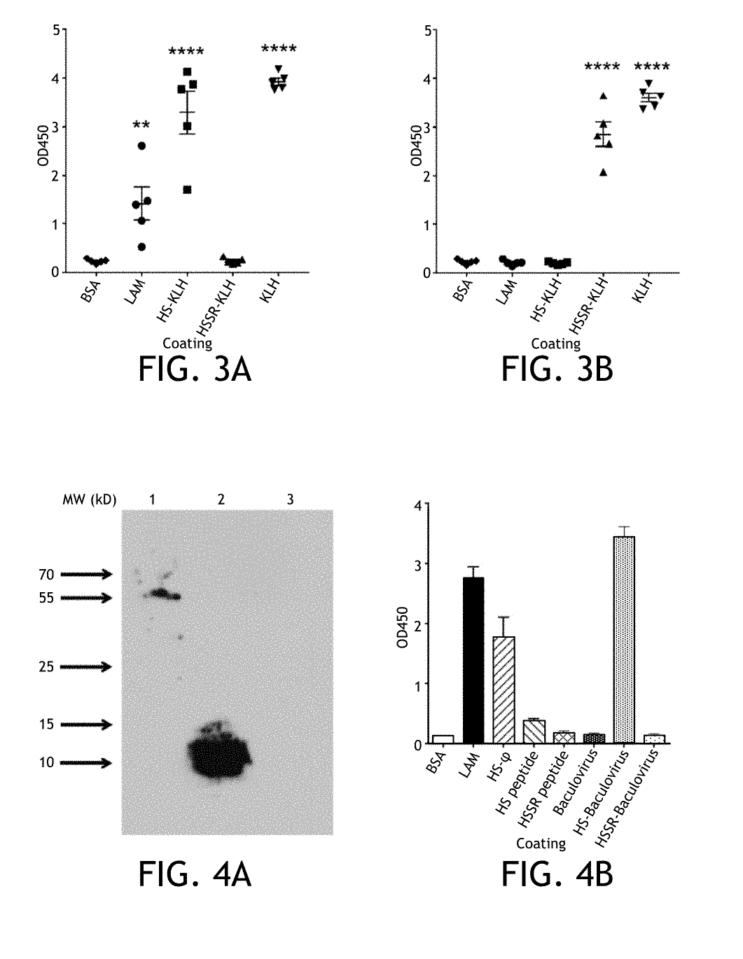

To confirm that the HS-peptide had mimotope activity in vivo, Swiss-Webster mice were vaccinated with KLH-conjugated HS284 peptide or KLH-conjugated HSSR peptide with alum adjuvant and their antibody responses were measured after 4 injections with KLH-conjugated mimotope peptides. All mice vaccinated with the HS peptide conjugated 286 to KLH generated antibodies against HS peptide and KLH but not the HSSR peptide or BSA, as expected (FIG. 3A). Likewise, all mice vaccinated with the HSSR peptide conjugated to KLH generated antibodies against HSSR peptide and KLH but not HS peptide or BSA (FIG. 3B) However, only mice vaccinated with the HS peptide conjugated to KLH developed antibodies against LAM, though there was significant variation in the response of individual mice (FIG. 3A). No HSSR-vaccinated mice generated antibodies reactive with LAM by ELISA (FIG. 3B). Taken together, the data show that the HS peptide can function as a mimotope in vivo when conjugated to the hapten KLH in the presence of an adjuvant.

### Example 5: Peptide Conjugation to Baculovirus

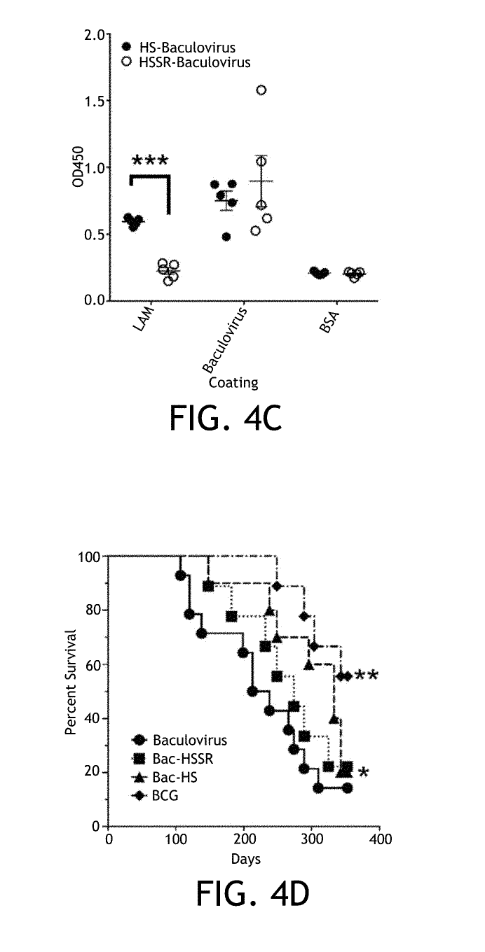

While in the process of biopanning for novel mimotopes, the ability to conjugate peptides successfully to baculovirus as has been previously reported was tested (Wilson et al., 2008). A biotinylated version of Peptide 1 (Table 1) was synthesized, conjugated to baculovirus and then the conjugation was tested by Western blot and ELISA. Both peptide conjugated baculovirus (FIG. 4A, lane 1) and free peptide (FIG. 4A, lane 2) could be detected with an anti-biotin antibody, but baculovirus alone (FIG. 4A, lane 3) could not.

### Example 6: Immunogenicity of Mimotope Conjugated Baculovirus

To test the ability of HS-peptide to be recognized as a LAM mimotope when conjugated to baculovirus, ELISA plates were coated with HS-peptide conjugated to baculovirus, the HSSR-peptide conjugated to baculovirus as well as controls and measured binding by anti-LAM ELISA. The anti-LAM antibody bound LAM, the HS-peptide conjugated to phage and also to baculovirus (FIG. 4B). Neither baculovirus alone nor HSSR-peptide conjugated baculovirus were detected by the anti-LAM antibody, demonstrating the specificity of the HS-peptide.

To further test the immunogenicity of the baculovirus conjugates as LAM mimotopes, mice were vaccinated intranasally with either HS-conjugated baculovirus or HSSR-conjugated baculovirus and evaluated the serum IgG response after 4 vaccinations. While both vaccinations yielded anti-baculovirus antibodies (FIG. 4C), only vaccination with the HS-peptide conjugated baculovirus induced an anti-LAM mimotope response (FIG. 4C). This mimotope activity was lower than the activity induced by KLH-conjugated HS peptide (compare FIG. 4C to FIG. 3A). In addition, no anti-LAM IgA antibodies could be detected in broncheoalveolar lavage of vaccinated mice (data not shown).

### Example 7: Effect of Vaccination on Mouse Survival

To test the ability of baculovirus conjugated HS-peptide to protect mice from tuberculosis, Swiss-Webster mice were vaccinated with either baculovirus alone, HS-conjugated baculovirus, HSSR-conjugated baculovirus or BCG as a positive control and then all of the mice were infected with 200 CFU of M. tuberculosis Erdman strain via aerosol. Vaccination with the HS-conjugated baculovirus partially protected mice from M. tuberculosis mortality 321 compared to baculovirus alone, though the protection was not as robust as vaccination with BCG (FIG. 4D). Despite the relatively modest mimotope response, vaccination with the HS-baculovirus did protect mice against M. tuberculosis mortality, though not as effectively as the established BCG vaccine. To that end, identification of mimotopes against other important cell wall antigens might provide even greater protection when combined with anti-LAM mimotopes.

### Example 8: Experimental Procedures

A. LAM Antibodies and Screening

Anti-LAM mouse monoclonal antibodies specific either for non-mannose-capped LAM, CS35 (Antibody class IgG3κ; NR-13811), or mannose-capped LAM (ManLAM), CS40 (Antibody class IgG1κ; NR-13812) and rabbit polyclonal rabbit anti-Mtb LAM antiserum (NR-13821) were generously provided by BEI Resources NIAID, NIH. To compare binding reaction of these antibodies with LAM, a LAM coating ELISA was performed. Five μg of LAM (NR-14848) provided by BEI Resources NIAID, NIH was used as a coating antigen following classic ELISA methods. Secondary anti-mouse and anti-rabbit antibodies conjugated to HRP were from Jackson ImmunoResearch Laboratories Inc. (catalogue #715-035-151 and 111-035-003, respectively). Detection of bound antibody complexes was with the 1-Step™ Ultra TMB-ELISA substrate solution (Thermo Scientific Co, Ltd; catalogue #34028).

B. Screening of Phage Displayed Peptide Library

Seven-mer (E8100s) and twelve-mer (E8110S) phage display peptide libraries (New England Biolabs) were panned with the selected mAb and titrated by plaque assay according to manufacturer's instructions. Briefly, a well of a 96-well microtiter plate was sensitized with 25 μg of selected mAb and incubated at 4° C. overnight. Blocking was performed with 350 μl of BSA (5 mg/ml) in 50 mM Tris-buffered saline (TBS) for 1 hour at room temperature followed by six washes with 50 mM TBS containing 0.1% Tween 20 (0.1% TBS-T) to remove excess antibody and blocking reagent. Approximately 2×1011 pfu recombinant phage were added to the well and incubated for 1 hour. Unbound phage were removed by repeat (10×) washing with 0.1% TBS-T. Antibody-bound phage were eluted by treatment with 100 μl of 0.2M Glycine-HCl (pH 2.2) followed by 15 μl of 1M Tris-Cl (pH 9.1) containing BSA. The eluted phage were amplified in log phase E. coli ER2738 and harvested in the culture supernatant at 12,000×g for 10 minutes 125 and concentrated by PEG/NaCl precipitation. Bio-panning was repeated 3 times on amplified phage eluate using a wash buffer containing 0.5% Tween 20 (0.5% TBS-T) to enrich the pool of phage with strong binding affinity to the mAb. Eluted and amplified phages were titrated after each round of panning.

C. Sequencing of mAb-Specific Phage

Following manufacturer's instructions, 10 phage colonies recovered after the fourth panning step were randomly selected and amplified. Each selected phage was excised from the bacterized agar and cultured in E. coli ER2738. Phage genomic DNA was extracted by treatment with Iodide buffer (pH 8.0) and purified by ethanol precipitation. The phage heptapeptide-gIII fusion gene was then sequenced and the corresponding amino acid sequence of the 7 or 12-mer peptide was deduced from the resulting nucleotide sequence using the reduced genetic code chart.

D. Peptide Synthesis

Peptides were synthesized and are summarized in Table 1. All peptides were synthesized with an N-terminal cysteine residue to permit chemical conjugation to KLH and baculovirus. Synthetic peptides were dissolved in distilled water and kept −20° C. until use.

E. Peptide ELISA Binding Assay

The ELISA plate wells were coated with by adding each peptide (2 μg in 0.1M NaHCO₃, pH 8.6) and incubating overnight at 4° C. After overnight incubation, plates were blocked with BSA (5 mg/ml) for 1 hour at 4° C. For controls, wells were coated with same amount of LAM or BSA. After washing with TBST, diluted 143 anti-LAM antibodies were added to the ELISA plate and incubated at room temperature for 1 hour. Plates were washed with TBST and HRP-conjugated secondary antibodies were added and incubated for 1 hour. After washing 3 times with TBST, 50 μl of the 1-Step™ Ultra TMB-ELISA (Thermo Scientific Co, Ltd) substrates was added in the dark. After color development, 50 μl of 2M sulfuric acid was added to stop the reaction and absorbance was measured at 450 nm.

F. KLH Conjugation

Peptide conjugation to KLH was performed using Imject Maleimide Activated Carrier Protein Kits (Thermo Scientific Co., Ltd; catalogue #77115) following the manufacturer's instructions.

G. Baculovirus Conjugation

Wild type AcMNPV (5×108 PFU/ml) was obtained from Kinnakeet Biotechnology (USA). AcMNPV conjugation with target peptides was performed using the Sulfo-SMCC (sulfosuccinimidyl 4-(N-maleimidomethyl)cyclohexane-1-carboxylate) reagent (Thermo Scientific catalogue #22322) following manufacturer's instructions as described (Wilson et al., 2008). Briefly, 4 mg/ml of Sulfo-SMCC crosslinking reagent was added to 10 mg AcMNPV in conjugation buffer and incubated for 30 minutes at room temperature. After desalting to remove unbound crosslinker, 10 μg of peptide was added to the SMCC crosslinker-coated AcMNPV and incubated for 20 minutes at room temperature followed by a desalting step to remove unbound peptide. The AcMNPV-peptide conjugates were then stored at −80° C. until use.

H. Mouse Vaccination to Measure Antibody Response

Swiss Webster outbred mice (5 mice/group, Taconic Biosciences) were vaccinated with PBS or mimotopes-conjugated to KLH or baculovirus. For mice receiving PBS, KLH alone or peptide-conjugates to KLH, inoculations were via subcutaneous injection. For the baculovirus groups, inoculations were intranasal. On day 0, serum from each mouse was collected from the retroorbital plexus (pre-immune), and 10 μg of peptide-conjugate or control was inoculated per animal. For testing mimotopes conjugated to KLH, vaccination also included alum adjuvant. Animals were boosted every two week interval for 3 additional times. Before each boost, serum was collected from each mouse and stored at −20° C. until use.

I. Mouse Serology

To perform ELISAS, 96-well ELISA plates (Greiner Bio-One Catalogue #655-001) were coated with 10 μg of corresponding coating antigens: HS peptide, HSSR peptide, Baculovirus, SG peptide, SGSR peptide, or LAM resuspended in coating buffer (0.1M NaHCO₃, pH 8.6). After overnight coating at 4° C., blocking (5% skim milk in PBS) was performed for 1 hour at room temperature. After washing 3 times with washing buffer (0.01% Tween-20 in PBS), mouse serum was added to the appropriate wells and binding proceeded at room temperature for 1 hour. Plates were washed 3 times with washing buffer (0.01% Tween-20 in PBS) and HRP177 conjugated anti-mouse IgG at 1:5,000 dilution (Jackson ImmunoResearch Laboratories Inc. catalogue #715-035-151) was added to each well. After incubating for 1 hour at room temperature, the plate was washed 3 times with TBST and 50 μl 179 of the 1-Step™ Ultra TMB180 ELISA substrate (Thermo Scientific) was added. 50 ul of 2M sulfuric acid was then added to stop the reaction and absorbance was measured at 450 nm.

J. Mouse Aerosol Infection and Survival Study

To test the ability of the anti-LAM mimotope to protect mice from subsequent tuberculosis infection, outbred Swiss Webster mice were first vaccinated according to the protocol described above. Mice receiving BCG Pasteur were vaccinated once subcutaneously (s.c.) with 106 bacteria in 0.2 ml PBS 8 weeks prior to receiving a M. tuberculosis Erdman challenge. After 3 booster injections, mice were then infected with 200 CFU of M. tuberculosis Erdman strain in a Glas-Col aerosolization chamber as described (Nair et al., Cell reports. 2016; 16:1253-8; Collins et al., Cell host & microbe. 2015; 17:820-8; and Zacharia et al., MBio. 2013; 4:e00721-13). On day 0, 5 unvaccinated mice were sacrificed to determine the initial CFU. Afterwards, mice were monitored weekly for weight loss and were euthanized when they had lost >15% of their body weight.

K. Statistical Analysis

Statistical analysis was performed using GraphPad Prism version 6.01. Two tailed unpaired Student's t test was used for single comparisons. Analysis of Variance (ANOVA) was used for experiments with multiple comparisons. Gehan-Breslow-Wilcoxon test was used for mouse survival experiments.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references recited in the application, to the extent that they provide exemplary procedural or other details supplementary to those set forth herein, are specifically incorporated herein by reference.

1-83. (canceled)

84. A method of eliciting an immune response against Mycobacterium tuberculosis or Mycobaterium bovis in a subject comprising administering to the subject an effective amount of a composition comprising a peptide of SEQ ID NO:1.

85. The method of claim 84, wherein the composition is formulated as a vaccine.

86. An isolated peptide having at least about 80% sequence identity or similarity to SEQ ID NO.:1.

87. The isolated peptide of claim 86, wherein the peptide is conjugated to a moiety, a carrier protein, or a vector.

88. The isolated peptide of claim 86, wherein the peptide is conjugated to a recombinant baculovirus.

89. The isolated peptide of claim 88, wherein the recombinant baculovirus is Autographa californica multinuclear polyhedrosis virus (AcMNPV).

90. The isolated peptide of claim 4, wherein the carrier protein is keyhole limpet hemocyanin (KLH) protein.

91. A vaccine composition comprising the isolated peptide of claim 86.

92. The vaccine composition of claim 46, further comprising an additional M. tuberculosis vaccine.

93. The vaccine composition of claim 47, wherein the additional M. tuberculosis vaccine is comprising a Bacillus Calmette Guerin (BCG) vaccine.

94. A pharmaceutical composition comprising the isolated peptide of claim 86.

95. A kit comprising a composition comprising the isolated peptide of claim 86.

96. A method of preparing an immunoglobulin to prevent or treat M. tuberculosis or M. bovis comprising the steps of immunizing a recipient with a composition comprising SEQ ID NO:1 and isolating the immunoglobulin from the recipient.

97. A method of detecting the presence of M. tuberculosis or M. bovis in a subject, the method comprising detecting anti-LAM antibodies in a sample obtained from the subject using a composition comprising SEQ ID NO:1 to determine the presence or absence of M. tuberculosis.

98. A lipoarabinomannan (LAM) peptide mimotope comprising SEQ ID NO:1, wherein the peptide mimotope is conjugated to a carrier or a vector.

99. The LAM peptide mimotope of claim 98, wherein the peptide is an anti-Mycobacterium tuberculosis LAM peptide mimotope.

100. The LAM peptide mimotope of claim 98, wherein the peptide is an anti-Mycobacterium bovis LAM peptide mimotope.

101. The peptide mimotop of claim 98, wherein the vector is a recombinant baculovirus.

102. The peptide mimotope of claim 101, wherein the recombinant baculovirus is Autographa californica multinuclear polyhedrosis virus (AcMNPV).

103. The peptide mimotope of claim 98, wherein the carrier is keyhole limpet hemocyanin (KLH) protein.
